(19) United States
(12) Patent Application Publication
(10) Pub. No.: US 2005/0128842 A1
(43) Pub. Date: Jun. 16, 2005

(54) Annular magnetic nanostructures

(76) Inventor: Alexander Wei, West Lafayette, IN (US)

Correspondence Address: BRINKS HOFER GILSON & LIONE, P.O. BOX 10395, CHICAGO, IL, 60610, US

(21) Appl. No.: 10/981,921

(22) Filed: Nov. 5, 2004

Related U.S. Application Data
(60) Provisional application No. 60/518,885, filed on Nov. 7, 2003.

(51) Int. Cl. C12P 21/04

(52) U.S. Cl. 365/205

(57) ABSTRACT

The present invention is directed to an integrated nanostructure with one or more electrically conductive nanowires and one or more annular magnetic nanostructures. Magnetic nanoparticles can form annular assemblies encircling the nanowire in the presence of a magnetic field produced by the passage of a current through the nanowire. The annular nanostructures support bistable magnetic flux closure states, which can be switched or polarized depending on the direction of the current through the nanowire.

### RELATED APPLICATION

This application claims the benefit of U.S. Provisional Application No. 60/518,885, filed Nov. 7, 2003, the entire contents of which are incorporated herein by reference.

### BACKGROUND

The present invention generally relates to nanostructures. More specifically, the invention relates to annular magnetic nanostructures.

Nanostructures with bistable magnetic states have exciting potential as nonvolatile memory elements for high-density information storage and as spin valves in magnetoelectronic devices. Magnetic nanostructures with annular geometries such as rings, toroids, and tubes are particularly intriguing candidates for storing binary data, because they are capable of supporting vortex-like domains known as flux closure (FC). These states have a net magnetostatic energy of zero, with self-contained induction and minimum stray magnetic flux beyond their outer perimeter. This latter feature suggests that adjacent FC states will not experience magnetic coupling, enabling the organization of magnetic elements into densely packed arrays with minimum crosstalk.

The concept of magnetic rings as memory core elements was first introduced in the early days of electronic data-storage applications, prior to the development of semiconductor-based memory. Devices were typically included millimeter-sized ferrite cores stitched onto electronic breadboards by copper wires, whose induction could be used for switching magnetic states. This primitive form of magnetic memory had obvious scaling limitations, and was later replaced by other types of magnetic recording media. However, these materials are now facing their own scaling limits, due partly to fabrication issues but also to read/write mechanisms which determine the reliability with which individual bits can be addressed. Such issues have sparked a surge of activity in magnetic nanomaterials, with the hope of identifying the means for further densification in nonvolatile data storage, as well as electronic processing mechanisms for faster data retrieval.

Interest in magnetic rings has been rekindled by a recent proposal that arrays of such rings could serve as individually addressable bits in the design of magnetic random-access memory (MRAM) devices. Extrapolation of this concept to nanoscale dimensions has obvious appeal, but one must first consider the fundamental limits of miniaturization to validate the feasibilility of operating at reduced length scales. These limits include the minimum size of the magnetic rings capable of supporting FC states, and the speed with which magnetic information can be recorded. The size limit for thermal stability of the FC state is dependent on the remanence of the magnetic material, which can be conveniently defined by the relationship KᵤV≧25 k_{B}T, where Kᵤ is the intrinsic magnetocrystalline anisotropy and V is the particle volume. As long as this criterion is met, magnetic nanorings made from high-Kᵤ materials should be capable of supporting remanent FC states at room temperature, down to diameters on the order of 10 nm. However, FC states in magnetic rings can also be generated by electrical currents passing through the center. In this case magnetic remanence is not an issue, so the unit particles can be decreased to sizes below the superparamagnetic limit of the host material.

With respect to the second fundamental limit, a research group has recently performed ultrafast switching experiments on granular CoCrPt films (t˜14 nm) which suggest the speed limit of magnetization reversal to be on the order of 10⁻¹² sec. This switching speed is still 2 to 3 orders of magnitude faster than the current state of the art in electronic data processing. Therefore, nanoring elements with magnetic FC states are excellent candidates for high-speed and high-density information storage and retrieval.

A number of research groups have developed lithographic approaches to fabricate such arrays, however in most cases the unit dimensions are in the submicron to micron range. Furthermore, many of these structures are produced by electron-beam lithography, a serial technique which is expensive and has low-throughput. Scalable approaches to lateral size reduction remain an outstanding challenge in materials fabrication, with direct impact on the maximum achievable areal densities.

Another critical issue in the development of magnetic ring arrays is their integration with electronic components for switching and reading magnetic states. This problem becomes increasingly challenging with size reduction: if a sequential, top-down approach is used, consistent registration between rings and wires is difficult to maintain, resulting in poor device reproducibility. Furthermore, a reduction in size will likely require the development of new read/write mechanisms which can circumvent the “interconnect problem” created by the differences in length scale at the macro/nano interface. Despite its importance, there are currently few published methods and no reported solutions which directly address this integration problem. Incremental advances in established technologies are thus unlikely to provide a solution.

### SUMMARY

In satisfying the above need, as well as overcoming the enumerated drawbacks and other limitations of the related art, the present invention provides annular magnetic nanostructures with FC states and methods for fabricating such nanostructures, and integrating them with electrically conductive nanowires. In various implementations, these annular magnetic nanostructures include (i) nanoparticle rings, created by dipole-directed self-assembly; (ii) continuous nanorings, created by templated synthesis; (iii) magnetic nanorings assembled around nanowire templates (nanorotaxanes); (iv) magnetic nanoparticle claddings assembled around nanowire templates; (v) magnetic core-shell nanowires, with electrically conductive cores encased in coaxial magnetic nanotubes; and (vi) any combination of the above. Materials which can support FC states can be magnetically soft or hard and electrically conductive or semiconductive, depending on the application. Similarly, the FC states can be of a nonvolatile nature (i.e. persist in the absence of an externally applied field) or be generated spontaneously by current-induced magnetic fields.

The materials and methods of this invention represent a departure from conventional top-down approaches for creating device architectures for magnetoelectronic and data storage applications. Moreover, the methodologies provided by various implementations of this invention can be combined with lithographic processes.

Further aspects, features and advantages of this invention will become readily apparent from the following description, and from the claims.

### BRIEF DESCRIPTION OF THE DRAWINGS

FIG. 1 illustrates self-assembly of ferromagnetic Co nanoparticles into nanorings, directed by magnetic dipolar interactions.

FIG. 2 illustrates stable FC states in Co nanorings as visualized by electron holography, with the arrows indicating the direction of magnetic flux.

FIG. 3 illustrates (a) a conducting nanowire producing a circular polarized magnetic field, (b) field-induced assembly of magnetic nanoparticles around a conducting nanowire, and (c) assembly after field exposure for an extended period, resulting in a densely packed cladding of magnetic particles.

FIG. 4a illustrates a side view of a nanorotaxane comprised of a magnetic nanoring and a conducting nanowire, inserted between two parallel nanowires to form an interdigitated magnetoresistive junction in accordance with the invention.

FIG. 4b illustrates a cross-sectional view of the interdigitated magnetoresistive junction of FIG. 4a.

FIG. 5a illustrates a side view of a nanorotaxane comprised of a magnetic nanoring and a magnetic core-shell nanowire, conjoined to a perpendicular nanowire to form a radial magnetoresistive junction in accordance with the invention.

FIG. 5b illustrates a cross-sectional view of the radial magnetoresistive junction of FIG. 5a.

### DETAILED DESCRIPTION

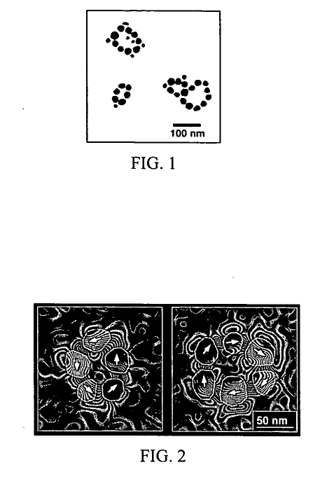

Self-assembly of nanoparticles have been demonstrated to be a useful alternative to lithography for fabricating annular magnetic nanostructures. For example, as shown in FIG. 1, weakly ferromagnetic Co nanoparticles can be fashioned into rings less than 100 nm across by dipole-directed self-assembly. As shown in FIG. 2, the magnetic dipoles within the Co nanoparticle rings align readily into FC domains as visualized by electron holography, a specialized electron microscopy technique, with the arrows indicating the direction of magnetic flux. These FC states are bistable at room temperature and persist in applied magnetic fields as high as 1000 Oe, suggesting that further reduction in size is possible. The FC polarization of the nanoparticle rings can be reversed by a strong out-of-plane magnetic pulse, which provides for a novel mechanism for magnetization switching, and enables the manipulation of FC states in annular nanostructures via applied magnetic fields.

A. Assembly of magnetic nanoparticle rings and claddings around nanowires.

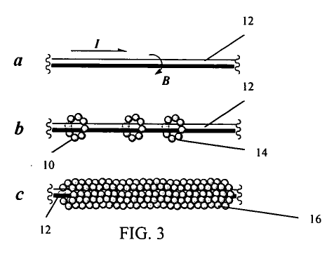

In accordance with the invention, magnetic nanoparticles 10 can be assembled around electrically conductive nanowires 12 into rings 14 and semicontinuous claddings 16, as shown in FIG. 3. Chemical structures with ring-and-axle topologies are commonly known as rotaxanes (see, e.g., Schiller, G., Catenanes, Rotaxanes, and Knots, Academic Press, New York, 1971, Vol. 22); hence, heterostructures with nanorings around nanowires are referred to here as nanorotaxanes. The assembly of magnetic nanoparticles into annular nanostructures can be mediated by magnetic dipolar interactions between nanoparticles, by dissipative forces driven by solvent evaporation, by changes in surface tension or interfacial surface energies, or any combination thereof. Magnetic dipolar interactions can be magnetostatic in nature or induced by local magnetic field gradients produced by the templating nanowire.

The magnetic nanoparticles are dispersed in the presence of electrically conductive nanowires, which can include any material capable of supporting electrical currents such as carbon nanotubes, metallic or semiconducting nanowires, or coaxial core-shell nanowires and/or nanotubes. The nanowires are connected to source and drain electrodes, which can be comprised of similarly conductive materials with variable configurations, including sharp metallic tips such as those used in scanning probe microscopy, interdigitated microelectrodes, macroscopic electrode surfaces having robust physical and electrical contact with the nanowires, electrodeposited electrodes on nanopatterned surfaces, conducting surfaces supporting nanoparticle catalysts for nanowire growth, or any combination thereof. The diameters of the nanowires are preferably in the range of 10 to 50 nm, but smaller or larger diameters may also be used depending on the material.

In one example of the invention, conductive nanowire arrays immersed in a suspension of superparamagnetic, ferrimagnetic, or ferromagnetic nanoparticles induce the coaxial assembly of magnetic nanorings or claddings upon passage of an electrical current. The nanoparticles can be comprised of any magnetically responsive material, including metals, alloys, or composites containing Cr, Mn, Fe, Co, Ni, Cu, or rare-earth elements such as the lanthanides (elements 58-71), as well as chalcogenides (e.g. oxides, sulfides, selenides), pnictides (e.g. nitrides, phosphides, arsenides), borides, carbides, or silicides of the above. The blocking temperature of the nanoparticles, T_{B}(a bulk property which provides a crude measure of the onset of magnetic responsivity), is preferably in the range of 250-350 K, but materials with lower or higher values for T_{B} may also be used. The medium is preferably a nonpolar organic liquid with a low boiling point for easy removal, but can also include polar or partially aqueous solvents. The dispersion can be performed with the aid of surfactants. These are preferably ones with macrocyclic headgroups and multiple tailgroups, such as structures based on tetra C-undecylcalix[4]resorcinarene (for example, see U.S. patent application Ser. No. 10/218,815, the entire contents of which are incorporated herein by reference), but can also include other chemisorptive surfactants, polymers, and polyelectrolytes.

The current densities passing through the nanowires for inducing magnetic nanoring assembly are preferably in the range of about 10⁻⁷ to 10⁻⁵ A/nm², but lower and higher current densities may also be used. The current passing through the nanowires produce generally circularly polarized magnetic flux according to Ampère's Law, B=μ₀//2πr, where μ₀ is the relative permeability (4π×10⁻⁷ Wb/A·m), / is the current, and r is the distance from the wire center. For example, if a current density of 10 μA/nm² is applied across a 50-nm Au wire, it produces a magnetic induction of about 1600 G at the metal surface and about 320 G at a 100-nm distance from the nanowire. This magnetic field gradient is capable of generally directing the formation of nanoparticle rings and claddings, with intraannular diameters determined by the nanowire templates.

B. Spin-polarized transport using magnetic nanorotaxanes and core-shell nanowires.

Further in accordance with the invention, conductive nanowires are prepared with continuous magnetic claddings for magnetoelectronics applications. Although there are reports of one-dimensional magnetic nanomaterials in the form of solid nanowires or hollow nanotubes, conductive nanowires sheathed in ultrathin magnetic layers have not been described in the prior art. In accordance with the invention, core-shell nanowires of this sort can be prepared in at least two ways. First, nanowires coated with magnetic nanoparticles can nucleate the chemical or electrochemical reduction of metal ions to produce a continuous coating. Magnetic nanoparticle coatings can be produced by field-induced self-assembly (cf. cladding 16), by chemical recognition mediated by chemisorptive surfactants, or by a combination of the above. Second, nanowires can be grown in conjunction with chemical vapor deposition (CVD) for heteroepitaxial core-shell growth, to produce conductive nanowires with coaxial magnetic nanotubes. For example, nonmagnetic nanowires with Si—Ge core-shell compositions have been prepared by this method, as described in Lauhon et al., Nature, 2002, 420, pp. 57-61, the entire contents of which are incorporated herein by reference.

Also in accordance with the invention, magnetic core-shell nanowires can support cylindrically stacked FC states, whose polarizations are spontaneously determined by the current direction. The coaxial shell can be (i) conductive or semiconductive; (ii) comprised of a magnetically soft material capable of responding to current-induced magnetic fields, and (iii) in ohmic contact with the core nanowire, which serves as a word line. This provides the foundation for spin-polarized transport across the magnetic cladding.

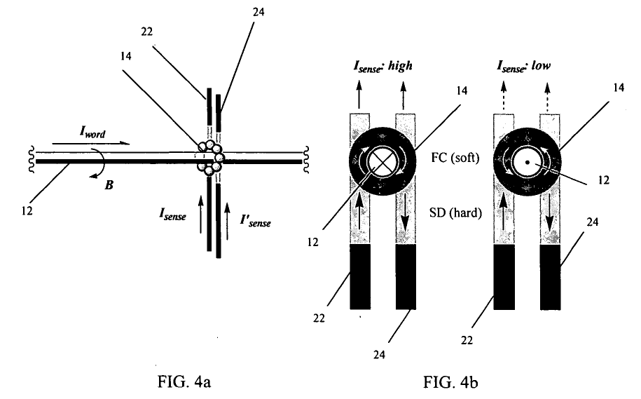

Further in accordance with the invention, additional nanowires can be integrated with preformed magnetic nanorings, nanorotaxanes, or core-shell nanowires and serve as address lines for reading FC states. Nanowires positioned next to magnetic nanostructures can produce out-of-plane magnetic fields for FC switching. For example, as shown in FIG. 4a, nanorotaxanes can be interdigitated between two conductive nanowires 22 and 24. Specifically, as shown in FIG. 4b, the coaxial nanowire 12 passes through the nanoring 14, and the two sensing nanowires 22 and 24 abut against the magnetic nanoring 14. The parallel nanowires can contain ferromagnetic domains, thereby serving as spin valves for spin-polarized transport. Thus, a nanosized magnetoresistive junction is formed by the interdigitation of a magnetic nanorotaxane (assembly of 12 and 14) between nanowires 22 and 24 having ferromagnetic domains, with nanorotaxane wire 12 serving as a word line for switching FC states, and nanowires 22 and 24 acting as sense lines.

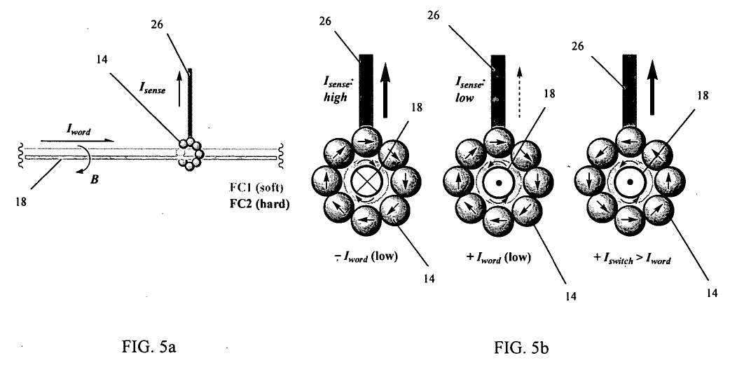

Yet also in accordance with the invention, a magnetoresistive junction is created by forming a second coaxial annulus around a magnetic core-shell nanowire 18. The outer layer can be a nanoparticle ring 14, a semicontinuous nanoparticle cladding 16, or a continuous coaxial layer. For example, as shown in FIGS. 5a and 5b, a nanowire 18 coated with a thin magnetic layer having minimal coercivity passes through a magnetic nanoring 14, which is connected to a second nanowire 26 extending radially from magnetic nanoring 14. The outer annulus is conductive or semiconductive and comprised of a magnetic material with nonzero coercivity, requiring a threshold current (/_{word}>/_{switch}) for magnetization reversal. That is, the inner nanowire 18 serves as a word line and can switch the FC state of the outer nanoring 14 at some current threshold /_{switch}. The radial nanowire 26 serves as a sense line and can carry relatively high currents when the inner and outer magnetic rings have the same FC polarization.

The inner and outer magnetic annuli can be separated by an ultrathin layer of dielectric or nonmagnetic metal such as Cu, similar to that used in magnetoresistive spin valves (see, e.g., Kanai, H et al., Fujitsu Sci. Tech. J., 2001, 37, pp. 174-182, the entire contents of which are incorporated herein by reference). The outer shell is in ohmic contact with additional conductive wires 26, which serve as sense lines. These wires can be grown radially from the outer magnetic nanoshell, such as by vapor-liquid-solid (VLS) synthesis. For example, controlled branching of nonmagnetic nanowires from a main nanowire ‘artery’ can be prepared by this method (see, e.g., Wang, D. et al., Nano Lett., 2004, 4, pp. 871-874, the entire contents of which are incorporated by reference).

The above and other embodiments of the invention are within the scope of the following claims.

1. A material comprising:
an electrically conductive nanowire;
one or more annular magnetic nanostructures which encircle the nanowire and are capable of supporting flux closure (FC) states, which are polarized or switched by the passage of an electrical current through the nanowire.

2. The material of claim 1 wherein the annular nanostructure is a nanoring, whose integration with the nanowire results in a nanorotaxane.

3. The material of claim 2 further comprising an additional pair of electrically conductive nanowires abutted against the nanorotaxane to produce out-of-plane magnetic fields for FC switching.

4. The material of claim 3 wherein the nanorotaxane is interdigitated between the pair of electrically conductive nanowires.

5. The material of claim 4 wherein the interdigitated conductive nanowires contain ferromagnetic domains in contact with the magnetic ring of the nanorotaxane, and serve as sense lines for spin-polarized transport.

6. The material of claim 1 wherein the annular nanostructure is a cladding.

7. The material of claim 1 wherein the nanowire is a carbon nanotube.

8. The material of claim 1 wherein the nanowire is metallic.

9. The material of claim 1 wherein the nanowire is a semiconducting nanowire.

10. The material of claim 1 wherein the nanowire has a coaxial core-shell structure.

11. The material of claim 10 wherein the core-shell structure is an inner wire sheathed in a magnetic layer.

12. The material of claim 11 further comprising a second annular structure surrounding the first annular structure and one or more additional conductive nanowires, the second annular structure being in contact with the one or more conductive nanowires, the additional conductive nanowires serving as sense lines.

13. A method of preparing nanostructures comprising:
immersion of at least one nanowire in a suspension of magnetically responsive nanoparticles; and
passage of an electrical current through the nanowire to create a magnetic field, such that the nanoparticles self-assemble into an annular nanostructure about the nanowire in the presence of the magnetic field, and a flux closure domain is created, whose polarization is induced by the direction of the current through the nanowire.

14. The method of claim 13 wherein the annular nanostructure is a semicontinuous or continuous nanoring.

15. The method of claim 13 wherein the annular nanostructure is a semicontiuous or continuous cladding.

16. The method of claim 15 wherein a second coaxial annular nanostructure forms about the first annular nanostructure.

17. The method of claim 16 further comprising placing one or more additional conductive nanowires in ohmic contact with the second annular nanostructure, to serve as sense lines.

18. The method of claim 17 wherein the additional conductive nanowires are grown radially from the second annular nanostructure.

19. The method of claim 13 further comprising interdigitating a nanorotaxane between a pair of additional conductive nanowires containing magnetic domains, such that the additional conductive nanowires serve as sense lines for spin-polarized transport.
